(19) United States
(12) Patent Application Publication
(10) Pub. No.: US 2015/0257782 A1
(43) Pub. Date: Sep. 17, 2015

(54) Adjustable Catheter System

(71) Applicant: Michael Chaim Cohn, Weston, FL (US)

(72) Inventor: Michael Chaim Cohn, Weston, FL (US)

(21) Appl. No.: 14/212,008

(22) Filed: Mar. 14, 2014

(51) Int. Cl. A61B 17/3207

(52) U.S. Cl. CPC A61B 17/320725

(57) ABSTRACT

In accordance with one embodiment, an adjustable catheter system from human circulatory system is provided. The system for blood clot and debris removal may have a first guide-wire for guiding a collapsible balloon. The first guide wire for guiding the collapsible balloon may have a first end and a second end. The first end of the guide-wire is connected to a first introducer. The first introducer provides mechanics for sealably introducing the first guide-wire into human circulatory system.

### FIELD OF THE INVENTION

The present invention relates generally to a catheter system. More so, an adjustable catheter system displaces an inflatable balloon along a body cavity to dislodge debris, while capturing the debris with a stationary expandable filter and outer lumen.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

A blood clot, or thrombus, that forms within a blood vessel may continue to grow, blocking off the blood supply to certain parts of the body and causing damage to tissues and organs. In some cases vascular debris formed during medical procedures are released into the system not only after, but even during, lower extremity orthopedic surgery, particularly (though not exclusively) knee and hip surgery.

The debris, referred to as emboli herein, typically includes thrombotic material and fat, forms during blood stasis and/or is released from the marrow as a result of its manipulation during surgery. In some patients, blood clots come from one site, dislodge, travel downstream, and lodge in relatively small vessels causing a blockage, or embolization. Untreated, a vascular blockage due to thrombosis or embolization can result in the loss of an organ or extremity, with potentially life-threatening consequences.

Patients with acute lower extremity ischemia or Critical limb ischemia or peripheral artery disease (PAD) are often the most difficult vascular patients to treat. Acute lower extremity ischemia due to vascular occlusion requires urgent treatment to prevent the need for amputation or possible loss of life. Rapid diagnosis is therefore vital, and computed tomographic angiography (CTA) has become a useful tool in evaluation of patients with this problem. Surgical bypass or thrombectomy, as well as interventional treatment with thrombolysis and, usually, adjunctive angioplasty and stent placement are standard therapies.

A contemporary method of treating Critical limb ischemia or advanced stage of PAD is mechanical thrombolysis. The mechanical thrombolysis disrupts the clot mechanically using either saline jets or, more recently, ultrasound waves. Saline jets dislodge the clot using the Bernoulli Effect. Ultrasound waves, emitted at low frequency, create a physical fragmentation of the thrombus.

Another contemporary procedure to treat blood clots is thrombectomy/embolectomy where the removal of a thrombus from a blood vessel, performed as emergency surgery to restore circulation to the affected part. During surgery a longitudinal incision is made into the blood vessel, and the clot is removed. However, most of the patients with the acute lower extremity ischemia are older. Thus, an invasive procedure requiring incisions may introduce additional complications to an already complex and time sensitive procedure. Hence, there exists a need for treating acute lower extremity ischemia using a less invasive systems and methods.

In view of the foregoing, it is clear that these traditional techniques are not perfect and leave room for more optimal approaches.

### SUMMARY OF THE INVENTION

This invention is directed to an adjustable catheter system for dislodging and capturing debris in a human circulatory system with an adjustable balloon and a stationary filter and outer lumen. In one embodiment, the adjustable catheter system may include a system for removing debris from a human circulatory system. The system may have a guide wire for guiding a collapsible and inflatable balloon. A balloon catheter with an inflatable balloon at its tip for carrying debris from the body cavity. The balloon catheter having a proximal end and a distal end such that the balloon being configured to extend and retract from the distal end. The proximal end connects to at least one introducer. The proximal end provides the mechanics for sealably catheterizing the guide wire into a body cavity. The body cavity may include, without limitation, a vein, an organ, and a urinary bladder.

The distal end of the balloon catheter may be attached to the balloon. The balloon is inflated with the fluid using a fill tube. The fill tube may also be used to drain the balloon while deflating the balloon. In one embodiment, the fill tube is located within an outer lumen the. For example, the collapsible balloon may inflate into one of a circular disc or oval shaped body.

The outer lumen provides a removable protective sheath for the balloon catheter and houses the balloon catheter and the guide wire and filter. The guide wire balloon catheter and filter being concentric to the outer lumen. The outer lumen may be composed of flexible, impermeable material. The outer lumen may also have a radio opaque marking allowing users to align the filter with the distal edge of the outer lumen.

The filter at its distal end may include a set of legs. In accordance with one embodiment, the filter legs may be narrowly spaced apart so as to restrict only the debris from passing through. In accordance with another embodiment, the filter may have a semi-permeable material connecting the set of legs. The semi permeable material may allow only plasma, such as blood, to pass through. In one embodiment, the balloon catheter comprises a plurality of flanges at the distal end limiting the distal movement of the filter. For example, the at least one introducer, an outer lumen, balloon catheter and the guide wire are concentric.

In accordance with one embodiment, the subject matter disclosed herein may include an outer lumen. The outer lumen houses the guide wire, balloon catheter such that the guide wire is movably housed inside the outer lumen. The lumen may be composed of flexible, impermeable material. The outer lumen may include a diameter of up to twenty-two French. Moreover, the outer lumen further comprises at least one introducer. Whereas, a drainage section on the outer lumen may include a rinse fluid inlet and a fluid drain outlet.

In accordance with one embodiment of the subject matter provided herein, an adjustable catheter system for dislodging and capturing debris in a human circulatory system with an adjustable balloon and a filter is provided, the catheter system comprising:
a sheath for carrying debris received from a body cavity, the body cavity comprising a vein, artery or graft in a human circulatory system, the debris comprising blood clots, the balloon catheter comprising a proximal end and a distal end;
a balloon for dislodging the debris from the body cavity, the balloon being configured to extend and retract from the distal end, the balloon comprising a substantially circular shape when inflated The balloon is directed through a outer lumen/sheath and may or may not allow a directing guide wire through it. The proximal end attached to at least one introducer, the at least one introducer being configured to displace the balloon along a longitudinal axis of the guide wire and the body cavity, the at least one introducer further being configured to inject a fluid into the balloon for inflating the balloon;
a filter, the filter comprising a distally facing, collapsible membrane having a wide distal end, and a narrow proximal end, the wide distal end defining a volume that is configured to receive the debris dislodged by the balloon, the wide distal end further defining a diameter that engages the body cavity, the filter comprising a permeable material for enabling plasma to flow between the guide wire and the body cavity
the filter further comprising a self-expanding material having a shape-memory alloy, the shape-memory alloy at least one of a nitinol, copper, aluminum, nickel, titanium, zinc, gold, iron, or any combination thereof; or comprising of a balloon expandable alloy such as steel and
an outer lumen composed of flexible impermeable material removably housing the guide wire, the outer lumen comprising a diameter up to twenty-two French, the outer lumen further comprising a drainage section, the drainage section comprising a rinse fluid inlet and a fluid drain outlet, the outer lumen further comprising a plurality of flanges at the distal end limiting the distal movement of the guide wire,
wherein, the balloon is displaced along the longitudinal axis of the body cavity, while the filter remains substantially stationary,
wherein the adjustable displacement of the balloon enables the adjustable catheter system to remain inside the body cavity during operation.

In a second aspect, the balloon is displaced along a longitudinal axis of the body cavity to dislodge debris from the body cavity. The filter and the outer lumen remain stationary. The filter remains just distal to the outer lumen/sheath over the wire and balloon catheter to capture the debris and enable the plasma to flow through, between the outer lumen and the body cavity.

In another aspect, the diameter of the present invention is relatively smaller than the prior art. For example, the outer lumen comprises a diameter of twenty-two French or less.

These and other advantages of the invention will be further understood and appreciated by those skilled in the art by reference to the following written specification, claims and appended drawings.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1A illustrates perspective views of a catheter system, in accordance with one embodiment.

FIG. 1B illustrates an inflated balloon extended from the balloon catheter and a filter in an expanded position, in accordance with an embodiment.

FIG. 2A illustrate perspective views of the catheter system dislodging and capturing debris.

FIG. 2B illustrates the expanded filter capturing the debris in accordance with an embodiment.

FIG. 2C illustrates the balloon retractably pulling the debris, in accordance with an embodiment.

FIG. 3 illustrates a detailed perspective view of a control wire and a filter, in accordance with an embodiment.

FIG. 4 illustrates a perspective view of a balloon in accordance with an embodiment.

FIG. 5 illustrates another perspective view of an adjustable catheter system in accordance with an embodiment.

FIG. 6 illustrates a flowchart diagram of a method for dislodging and capturing debris in a human circulatory system with an adjustable balloon and a filter, in accordance with an embodiment.

### DETAILED DESCRIPTION OF THE INVENTION

The present invention is best understood by reference to the detailed figures and description set forth herein.

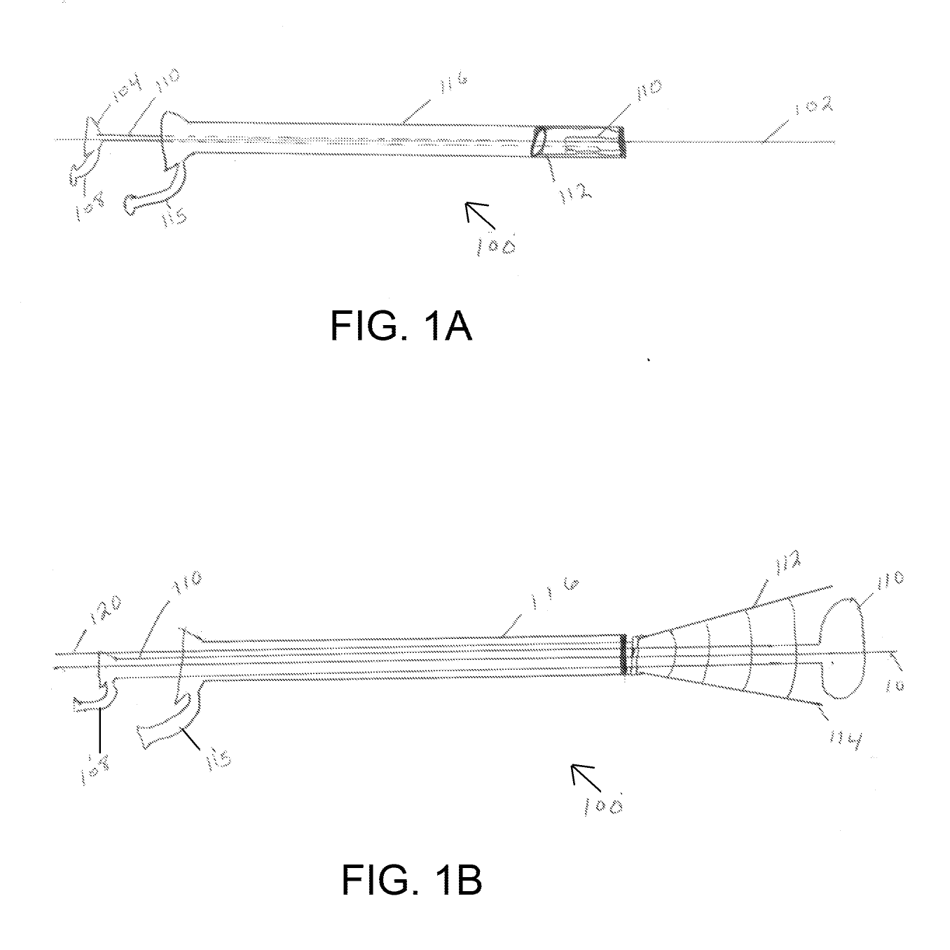

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

As referenced in FIGS. 1A and 1B, an adjustable catheter system 100 may at least partially remove a debris 122 from a human circulatory system. The system 100 may have a guide wire 102 for guiding a collapsible and inflatable balloon 110. The guide wire 102. FIG. 1B illustrates a balloon catheter 120 with an inflatable balloon 110 at its tip for carrying debris from the body cavity. The balloon catheter may include a proximal end 104 and a distal end 106 . . . For example, the balloon is attached at the distal end of the balloon catheter and the balloon catheter is configured to extend and retract from the distal end.

The proximal end 104 connects to at least one introducer 108. The proximal end 104 provides the mechanics for sealably introducing the guide wire 102, the balloon catheter 120 into a body cavity (not shown). The body cavity may include, without limitation, a vein, an organ, or a urinary bladder, or the like. An outer lumen 116 provides a removable protective housing the balloon catheter 120, and the guide wire 102. The guide wire 102, the balloon catheter 120, and the outer lumen 116 are substantially concentric to the body cavity. Suitable materials for fabricating the adjustable catheter system 100 may include, without limitation, polymers, silicone rubber, nitinol, nylon, polyurethane, and polyethylene terephthalate (PETE) latex, and thermoplastic elastomers.

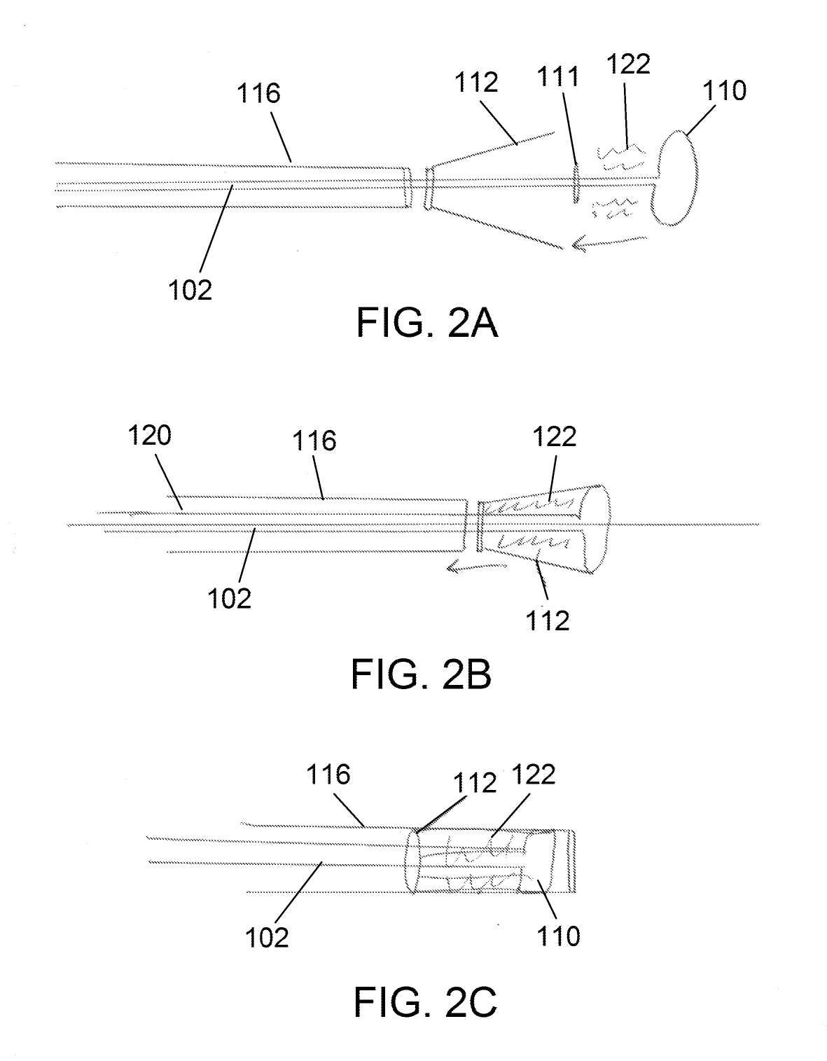

In some embodiments, the balloon catheter 120 may be retractable independent of the outer lumen 116. Optionally, the balloon catheter 120 may be retractable until the balloon 110 is submerged in the filter 112 and the outer lumen 116. Additional retraction of the balloon catheter 120 causes the balloon 110 and filter 112 to retract together, as referenced in FIG. 2C. In addition a control wire 120 may extend through the outer lumen 116, helping to control the filter 112.

The distal end 106 of the balloon catheter 120 may be attached to the balloon 110. The balloon 110 is inflated with the fluid using at least one introducer 108. The introducer 108 may be advanced as needed to place the balloon 110 in a desired position along the body cavity or the guide wire 102. The at least one introducer 108 comprises a fill tube 115. The fill tube 115 may be used to drain the balloon 110 while deflating the balloon 110. In one embodiment, the fill tube 115 is located within the balloon catheter 120. Once in place, the balloon 110 is inflated with the fluid. For example, the collapsible balloon 110 may inflate into one of a circular disc or oval shaped body.

Turning now to FIGS. 2A, 2B, and 2C, the balloon 110 is displaced along a longitudinal axis of the body cavity to dislodge debris 122 from the body cavity. The debris 122 may include, without limitation, blood clots, fat, and tissue. FIG. 2A illustrates an inflated balloon 110 engaging the debris 122, and extended out from the outer lumen 116. FIG. 2B illustrates the expanded filter capturing the debris 122 and retracting back into the outer lumen 116. FIG. 2C illustrates the balloon catheter 120 retractably pulling the debris 122 through the outer lumen 116. The debris 122 may then be discarded through a drain 124 on the proximal end 104.

Once extended out in a desired position, as referenced in FIG. 2A, the balloon 110 is inflated or deflated via the fill tube 115, thus pushing the balloon 110 against the inner lining of the body cavity. In one embodiment, the filter 112 remains substantially stationary. The body cavity may include living tissue, such as human or animal vein, artery, or duct. The balloon 110 is inflated to a pressure ranging preferably from approximately 3 to 15 atmospheres. The proper pressure will be dependent on the treatment protocol, the type of organism being treated, and the material from which the balloon 110 is constructed. The balloon 110 acts as one of a particle removal mechanism. The balloon 110 forms a trap/barrier such that, when it is retracted, the debris are trapped between the filter and balloon. As the debris are swept into the filter, the balloon and the filter are simultaneous removed thereby removing the trapped debris . . . ?

In one embodiment of the present system 100, an outer lumen 116 houses the guide wire 102, the balloon catheter. The outer lumen 116 may be composed of flexible, impermeable material. The diameter of the present invention is relatively smaller than the prior art. For example, the outer lumen 116 comprises a diameter of up to twenty two French. The outer lumen 116 may also have a radio opaque marking allowing users to align the filter 112 with the edge of the outer lumen 116.

The filter 112 at the distal end 106 of the balloon catheter 120 may include a set of legs 114. In accordance with one embodiment, the filter 112 may have a permeable material connecting the set of legs 114. The semi permeable material may allow only plasma, such as blood, to pass through. In one embodiment, the outer lumen 116 comprises a plurality of flanges at the distal end 106 limiting the distal movement of the filter 112. The filter 112 may include a shape-memory composition, such as nitinol or be balloon expandable such as stainless steel. In this manner, the filter 112 may expand against the inner liner of the body cavity at predetermined parameters, such as temperature, pressure, surrounding liquid viscosity.

In accordance with one embodiment, the subject matter disclosed herein may include an outer lumen 116. The lumen 116 may be composed of flexible, impermeable material. The outer lumen 116 may include a diameter of up to twenty-two French. Moreover, the outer lumen 116 further comprises at least one introducer 108. Whereas, the drain 124 on the outer lumen 116 may include a rinse fluid inlet and a fluid drain outlet.

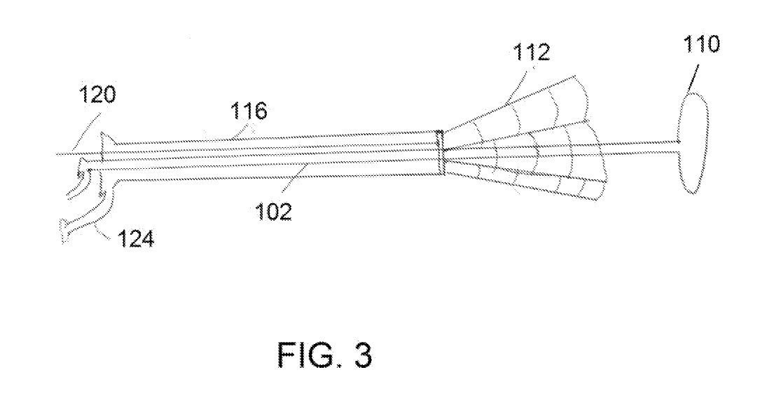

FIG. 3 illustrates a detailed perspective view of an alternative embodiment of the adjustable catheter system 100, where the filter 112 is connected to an outer lumen/sheath 116 or a control wire 120 may be connected to the filter 112, and regulates the filter 112 accordingly. The junction formed with the outer lumen 116 may help stabilize the control wire 120 and the filter 112.

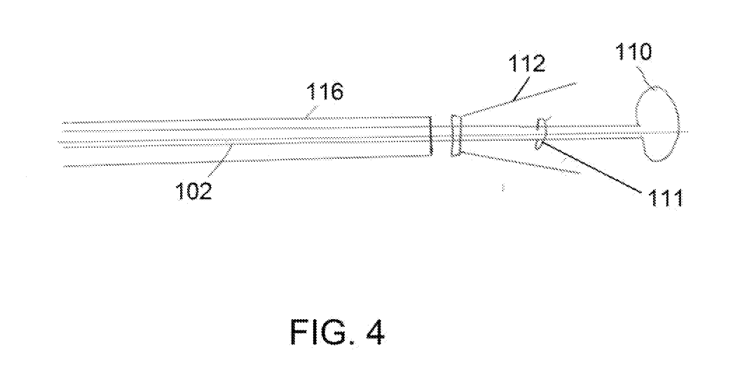

FIG. 4 illustrates a detailed perspective view of an alternative embodiment of the adjustable catheter system 100, where a balloon cuff 111 attaches to the balloon 110. The balloon cuff 111 forms a peripheral border that allows expansion and retraction of the filter in and out of the outer sheath.

The embodiment of the balloon catheter with a cuff 111 is configured to allow the balloon and the filter slide in and out of the sheath. The system comprises a balloon, a filter and an introducer such that the balloon and the filter are enclosed within the introducer. The introducer help squeeze the balloon and the filter into the sheath. The introducer carries the balloon and the filter up till the distal end of the sheath. As the balloon catheter is moved further distally, the balloon and the filter are unpacked from the introducer. The self-expanding filter conforms to the body cavity and the balloon is inflated to capture the debris.

As the balloon is retracted back into the sheath, the cuff engages the proximal end of the filter. The filter encloses the debris and the balloon as it moves back into the sheath. The balloon, filter, and the introducer are all extracted from the sheath/outer lumen and the captured debris removed or washed off. The filter and the balloon are manually placed/reinserted into the introducer and the procedure is repeated to remove further debris.

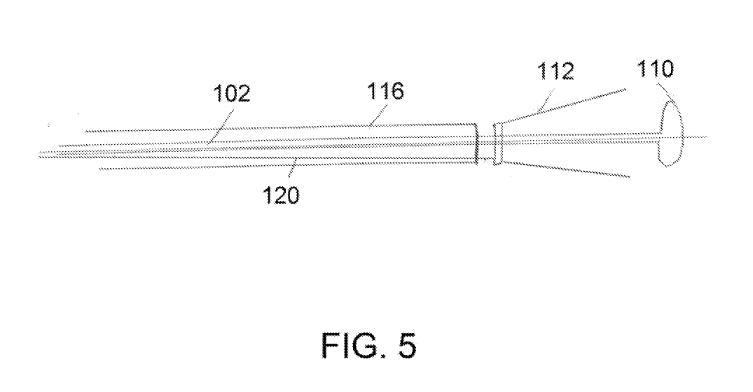

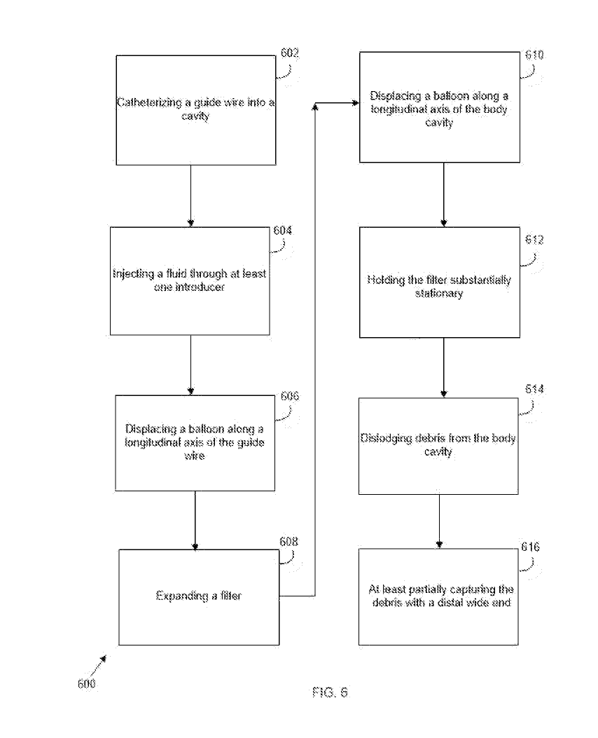

FIG. 5 illustrates a detailed perspective view of an alternative embodiment of the adjustable catheter system 100, where a control wire 120 attaches to a filter 120. The control wire 120 helps regulate the expansion and retraction of the filter 112. The control wire 120 may work in conjunction with the balloon catheter 120, where the balloon catheter 120 manipulates the balloon 110, and the control wire 120 manipulates the filter 112 accordingly.

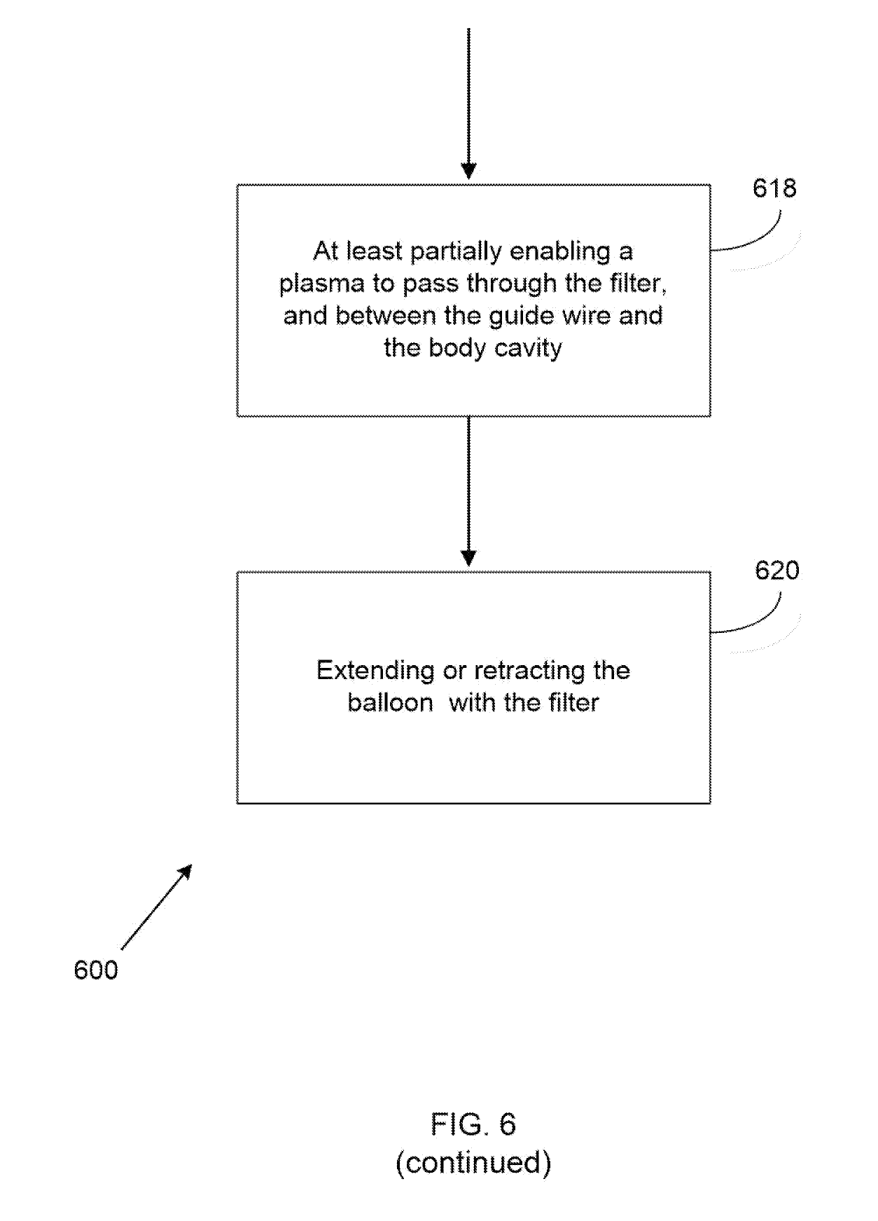

FIG. 6 illustrates a method 600 for dislodging and capturing debris 122 in a human circulatory system 100 with an adjustable balloon 110 and a filter 112. The method 600 may include an initial Step 602 of catheterizing a guide wire 102 into a body cavity. The outer lumen is subsequently directed over the wire to the required area in the vessel. The outer lumen up to twenty-two French in size is used to house a balloon catheter and filter system. The outer sheath is also used as a reservoir for removed debris. The balloon catheter 120 is advanced over the guide wire 102 through the outer lumen into the body cavity and filter 114 is advanced over the wire 102 and over the balloon catheter into the vessel. Filter 114 is deployed in the vessel held in place by distal legs 114 touching and circumventing the inner lumen of the vessel or by wire/s 120 attached to the proximal end of the filter 114. Filter 114 can be advanced so the proximal tip remains in the sheath 116 or is part of the sheath 116 and distal legs 114 expanded into vessel fully deployed with proximal and distal filter in the vessel over the balloon 110. Balloon 110 is advanced through the filter 114 over the wire 102 distal to the clot/thrombus. Balloon 110 is inflated with liquid until the outer portion of the balloon comes contact with the inner vessel lumen and pulled back toward the filter 114 carrying the clot/debris with it. The clot is thus trapped by the filter 114 as the balloon 110 is pulled into it. The filter 114 may be held in place by a wire 120 connected to its proximal end or without a wire held in place by the force of the legs 115 against the inner lumen of the vessel wall. The trapped clot is either retracted by the balloon 110 through the filter into the sheath 116 and removed through the sheath 116 by the drain 115 or pulled out with balloon through the proximal end of the sheath 116. Alternatively, the clot/debris is removed by the balloon 110 into the filter 112 and the entire balloon filter system extracted simultaneously securing the clot thus removing into the sheath 116 and out of the body.

In one embodiment a cuff 222 on the balloon catheter 110 retracts the filter and the balloon 110 together in the sheath 116 with the trapped clot is removed from the body. When the balloon catheter 110 is retracted from the vessel the cuff 222 pulls the ring on the proximal part of the filter with it retracting the filter 112, the balloon 110 and the clot simultaneously into the sheath 116 for removal.

In one embodiment a wire/s 120 can be attached to the proximal filter 112 and can stabilize and facilitate retraction of the filter into the sheath with clot or after clot has been extracted. In one embodiment the filter 112 can actually be part balloon catheter 120.

The balloon catheter 120 may be retractable until the balloon 110 is submerged in the filter 112. In one embodiment, any additional retraction of the balloon catheter 120 causes the filter 112 and the balloon 110 to retract together, as referenced in FIG. 2C.

In some embodiments, a Step 604 may include injecting a fluid through at least one introducer 108. The fluid may include a gas. The method 600 may then proceed to a Step 606 of displacing a balloon 110 along a longitudinal axes of the guide wire 102. The balloon 110 is inflated with the fluid using at least one introducer 108. The at least one introducer 108 may be advanced as needed to place the balloon 110 in a desired position along the guide wire 102. The at least one introducer 108 comprises a fill tube 115 (not shown). The fill tube 115 may be used to drain the balloon 110 while deflating the balloon 110. In one embodiment, the fill tube 115 is located within the balloon catheter 120. Once in place, the balloon 110 is inflated with the fluid. For example, the collapsible balloon 110 may inflate into one of a circular disc or oval shaped body.

In some embodiments, a Step 608 comprises expanding a filter 112. The filter serves to capture the debris 122, but still allow the plasma to pass through. The filter 112 may include a set of legs 114 connected by a permeable membrane. A Step 610 may include displacing the balloon 110 along a longitudinal access of the body cavity. The balloon 110 is displaced along a longitudinal axis of the body cavity to dislodge debris 122 from the body cavity. Once in a desired position, the balloon 110 is inflated or deflated via the fill tube 115, thus pushing the balloon 110 against the inner lining of the body cavity. In some embodiments, Step 612 comprises holding the filter 112 substantially stationary. A Step 614 may include dislodging debris 122 from the body cavity. The balloon 110 acts as one of a particle removal mechanism. The balloon 110 forms a trap/barrier such that, when it is retracted, the particles are trapped and removed.

Step 616 may include at least partially capturing the debris 122 with a distal wide end. The membrane allows the plasma to pass through, but funnels the debris 122 into the filter 112. A Step 618 comprises at least partially enabling plasma to pass through the filter 112, and between the outer lumen 116 and the body cavity. A final Step 620 comprises extending or retracting the balloon 110. The balloon 110 may be extended further into the body cavity while keeping the guide wire 102 and the outer lumen 116 stationary. In this manner, the adjustable catheter system 100 does not have to be removed from the body cavity during operation. Those skilled in the art will recognize that minimizing the steps and reducing friction on the body cavity increases the odds of a successful operation, and reduces the odds for infections.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What I claim is:

1. An adjustable catheter system for dislodging and capturing debris in a human circulatory system, the adjustable catheter system comprising:
a guide wire for guiding catheter within a body cavity, the guide wire being generally concentric to the body cavity;
a balloon catheter with an inflatable balloon at its tip for carrying debris from the body cavity, the balloon catheter comprising a proximal end and a distal end, the balloon being configured to extend and retract from the distal end,
the proximal end attached to at least one introducer, the at least one introducer being configured to displace the balloon along a longitudinal axis of the guide wire and the body cavity, the at least one introducer further being configured to inject a fluid into the balloon for inflating the balloon; and
a filter, the filter comprising a distally facing, collapsible membrane/phalanges having a wide distal end, and a narrow proximal end, the wide distal end defining a volume that is configured to receive the debris dislodged by the balloon, the wide distal end further defining a diameter that engages the body cavity,
wherein, the balloon is displaced along the longitudinal axis of the body cavity, while the filter remains substantially stationary,
wherein the adjustable displacement of the balloon enables the adjustable catheter system to remain inside the body cavity during operation.

2. The system of claim 1, wherein the body cavity further comprises a vein, and the debris further comprises a blood clot.

3. The system of claim 1 further comprises a control wire, the control wire configured to help control the filter.

4. The system of claim 1 further comprises an outer lumen composed of flexible impermeable material removably housing the guide wire, the filter, and the balloon catheter.

5. The system of claim 4, wherein the outer lumen comprises a radio opaque mark, a radiation opaque mark, or the like for aligning the filter during a medical procedure.

6. The system of claim 5, wherein the outer lumen is up to twenty-two French in diameter.

7. The system of claim 6, wherein the outer lumen comprises a drainage section, the drainage section comprises a rinse fluid inlet and a fluid drain outlet.

8. The system of claim 7, wherein the outer lumen comprises a plurality of flanges or other limiting mechanism at the inner edge on the distal end, the filter having a plurality of flanges or other limiting mechanism at the outer edge on the proximal end where by the proximal end of the filter engaging the distal end of outer lumen thereby limiting the distal movement of the filter.

9. The system of claim 1, wherein the filter comprises a self-expanding material having a shape-memory alloy.

10. The system of claim 1, wherein the outer lumen or the sheath having an expandable filter at the distal end.

11. The system of claim 1, wherein the filter comprises a plurality of legs.

12. The system of claim 11, wherein the plurality of legs spacing configured to capture the debris.

13. The system of claim 1, wherein the filter comprising a permeable material configured to connect the plurality of legs, the permeable material enabling plasma to flow through.

14. The system of claim 1, wherein the catheter comprises proximal end comprises a ring limiting distal movement of the filter.

15. The system of claim 1, wherein the balloon inflates into a circular disc or an oval shaped body.

16. The system of claim 1, wherein the at least one introducer sealably introduces the balloon catheter within the human circulatory system.

17. The system of claim 1, wherein the fluid is a liquid, semi-liquid, gas, or the like.

18. The system of claim 1 further comprises an combination introducer, the balloon and the filter enclosed within the combination introducer, the combination introducer help squeeze the balloon and the filter into the sheath, the introducer carrying the balloon and the filter up till the distal end of the sheath.

19. The system of claim 1 further comprises a cuff on the balloon catheter, the cuff engages the proximal end of the filter retracting the filter into the sheath as the balloon is retracted back into the sheath.

20. A method for at least partially dislodging and capturing debris in a human circulatory system, the method comprising:
providing an adjustable balloon and a filter system;
enabling the system to house the balloon and the filter within an outer lumen;
means for catheterizing a balloon and a filter into a body cavity;
means for injecting a fluid through at least one introducer to inflate the balloon;
means for displacing a balloon along a longitudinal axes of the guide wire;
means for expanding a filter;
means for holding the filter substantially stationary;
means for dislodging debris from the body cavity using the longitudinal movement of the inflatable balloon;
means for at least partially capturing the debris with a distal wide end of the filter;
means for at least partially enabling a plasma to pass through the filter, and between the guide wire and the body cavity; and
means for extending or retracting the balloon with the filter without removing the outer lumen from the body.
